(19) United States
(12) Patent Application Publication
(10) Pub. No.: US 2023/0080878 A1
(43) Pub. Date: Mar. 16, 2023

(54) APPLICATION OF TRADITIONAL CHINESE MEDICINE COMPOSITION IN PREPARATION OF DRUGS FOR TREATING OR PREVENTING CORONAVIRUS INFECTION

(71) Applicant: JIANGSU KANION PHARMACEUTICAL CO., LTD., Lianyungang, Jiangsu (CN)

(72) Inventors: Wei XIAO, Lianyungang (CN); Liang CAO, Lianyungang (CN); Zhenzhong WANG, Lianyungang (CN); Chenfeng ZHANG, Lianyungang (CN); Xinzhuang ZHANG, Lianyungang (CN); Zeyu CAO, Lianyungang (CN)

(73) Assignee: JIANGSU KANION PHARMACEUTICAL CO., LTD., Lianyungang, Jiangsu (CN)

(21) Appl. No.: 17/800,693

(22) PCT Filed: Dec. 16, 2020
(86) PCT No.: PCT/CN2020/136686

§ 371 Date: Aug. 18, 2022

(30) Foreign Application Priority Data
Feb. 21, 2020 (CN) 202010108894.1

(51) Int. Cl. A61K 35/32; A61P 31/14; A61K 33/06; A61K 35/413; A61K 36/484; A61K 36/539; A61K 36/708; A61K 36/8966

(52) U.S. Cl. CPC A61K 35/32; A61P 31/14; A61K 33/06; A61K 35/413; A61K 36/484; A61K 36/539; A61K 36/708; A61K 36/8966; A61K 2236/331; A61K 2236/39; A61K 2236/53

(57) ABSTRACT

An application of a traditional Chinese medicine composition in preparing a medicine for treating or preventing coronavirus infection. The traditional Chinese medicine composition is characterized by being prepared from the following raw materials in parts by weight: 5-15 parts of Cornu saigae tataricae or 50-150 parts of goral horns, 10-60 parts of Bulbus Fritillariae Ussuriensis, 15-60 parts of Radix et Rhizoma Rhei, 7-30 parts of Radix scutellariae, 7-30 parts of Lapis Chloriti, 10-50 parts of gypsum fibrosum, 5-20 parts of calculus bovis artifactus, and 15-60 parts of radix glycyrrhizae. The traditional Chinese medicine composition can inhibit 2019-nCoV replication in cells, so that cells infected by 2019-nCoV viruses have no lesion. Therefore, the traditional Chinese medicine composition can treat or prevent coronavirus related diseases.

### TECHNICAL FIELD

The present application relates to the field of disease treatment medicines, and in particular, to an application of a traditional Chinese medicine composition in preparing a medicine for treating or preventing coronavirus infection.

### BACKGROUND

2019-nCoV belongs to a β-coronavirus, which has an envelope and round or oval particles with a diameter of 60-140 nm, and is often polymorphic. A genetic characteristic of the 2019-nCoV is significantly different from that of SARS-CoV and MERS-CoV. Current research has shown that it has a homology of more than 85% with bat SARS-like coronavirus (bat-SL-CoVZC45). Respiratory droplets and close contact transmission are main routes of transmission. There is a possibility of aerosol transmission in a relatively closed environment for a long time exposure to a high-concentration virus aerosol. A patient is mainly manifested by a fever, fatigue, and a dry cough. A few patients have symptoms such as nasal congestion, runny nose, and diarrhea. In many severe patients, breathing difficulty occurs after one week, and those critical cases can quickly progress to acute respiratory distress syndrome, septic shock, and hardly corrected metabolic acidosis and coagulation dysfunction.

A jinzhen oral liquid in the present application includes the following raw materials: cornu saigae tataricae or goral horns, Bulbus Fritillariae Ussuriensis, Radix et Rhizoma Rhei, radix scutellariae, Lapis Chloriti, gypsumfibrosum, calculus bovis artifactus, and radix glycyrrhizae. The main functions of the oral liquid are clearing away heat and toxic materials, and expelling phlegm to arrest coughing. Some children with acute bronchitis as well as sputum and heat cough are manifested by a fever, a cough, yellow sputum in coughing or vomiting, a dry cough, a red tongue, a yellow and greasy tongue coating, and the like. It has a good improvement effect on respiratory diseases caused by various viruses and bacteria. Whether it can effectively prevent and treat coronavirus-related diseases, especially COVID-19, is a scientific research to be developed.

### SUMMARY

In view of this, the present application aims to provide an application of a traditional Chinese medicine composition in preparing a medicine for treating or preventing coronavirus infection. The traditional Chinese medicine composition is characterized by being prepared from the following raw materials in parts by weight: 5-15 parts of cornu saigae tataricae or 50-150 parts of goral horns, 10-60 parts of Bulbus Fritillariae Ussuriensis, 15-60 parts of Radix et Rhizoma Rhei, 7-30 parts of radix scutellariae, 7-30 parts of Lapis Chloriti, 10-50 parts of gypsum fibrosum, 5-20 parts of calculus bovis artifactus, and 15-60 parts of radix glycyrrhizae.

Further, diseases infected by the coronavirus include COVID-19 (novel coronavirus pneumonia).

Further, the traditional Chinese medicine composition includes: 8-12 parts of cornu saigae tataricae or 80-120 parts of goral horns, 15-55 parts of Bulbus Fritillariae Ussuriensis, 20-35 parts of Radix et Rhizoma Rhei, 10-18 parts of radix scutellariae, 10-18 parts of Lapis Chloriti, 15-25 parts of gypsum fibrosum, 8-12 parts of calculus bovis artifactus, and 20-35 parts of radix glycyrrhizae.

Further, the traditional Chinese medicine composition includes: 8-10 parts of cornu saigae tataricae or 80-100 parts of goral horns, 40-50 parts of Bulbus Fritillariae Ussuriensis, 25-35 parts of Radix et Rhizoma Rhei, 12-18 parts of radix scutellariae, 12-18 parts of Lapis Chloriti, 20-25 parts of gypsum fibrosum, 8-10 parts of calculus bovis artifactus, and 30-35 parts of radix glycyrrhizae.

Preferably, the traditional Chinese medicine composition includes: 9.45 parts of cornu saigae tataricae, 17.25 parts of Bulbus Fritillariae Ussuriensis, 31.5 parts of Radix et Rhizoma Rhei, 15.75 parts of radix scutellariae, 15.75 parts of Lapis Chloriti, 23.62 parts of gypsum fibrosum, 9.45 parts of calculus bovis artifactus, and 31.5 parts of radix glycyrrhizae.

Preferably, the traditional Chinese medicine composition includes: 94.5 parts of goral horns, 47.25 parts of Bulbus Fritillariae Ussuriensis, 31.5 parts of Radix et Rhizoma Rhei, 15.75 parts of radix scutellariae, 15.75 parts of Lapis Chloriti, 23.62 parts of gypsum fibrosum, 9.45 parts of calculus bovis artifactus, and 31.5 parts of radix glycyrrhizae.

Specially, the medicine for treating or preventing coronavirus infection includes an oral dosage form, an injection administration form or an external administration preparation.

Specially, the medicine for treating or preventing coronavirus infection includes a decoction, a tablet, a capsule, a granule, a pill, an injection, a decocted extract, a suspending agent, a dispersing agent, syrup, a suppository, a gel, aerosol, a patch or an oral liquid.

The raw materials used in the present application can be directly ground into powder, or may be extract prepared through conventional means in the art. The composition can be obtained by directly grinding several compositions into powder through a conventional method, or can be extract prepared through conventional means, so that different dosage forms are prepared, and therefore, a modern dosage form which has a better curative effect, and is more beneficial for being prepared is obtained.

Preferably, a preparation method for the traditional Chinese medicine composition includes the following steps: taking and cutting cornu saigae tataricae or goral horns into threads, hydrolyzing the threads with an alkaline-containing aqueous solution, filtering, and taking and concentrating filtrate; taking and smashing Lapis Chloriti and gypsum fibrosum into crude powder, heating and decocting the crude powder in water, filtering, and taking and concentrating filtrate; taking and extracting calculus bovis artifactus with 70% alcohol, filtering, and concentrating filtrate; decocting the rest of medicines in water, concentrating and centrifuging filtrate, and precipitating liquid supernatant with alcohol, standing, taking the liquid supernatant, filtering, and concentrating filtrate; and mixing the concentrated filtrate to obtain the traditional Chinese medicine composition.

More specifically, the composition is extracted by adopting the following method:

Cutting the cornu saigae tataricae or goral horns into threads, using the alkaline-containing aqueous solution to perform hydrolysis reflux twice, combining and concentrating filtrate obtained twice filtrate; smashing Lapis Chloriti and gypsum fibrosum into crude powder, heating and decocting the crude powder in water twice, filtering, combining filtrate obtained twice, and concentrating the combined filtrate to have a relative density of 1.01 to 1.04 at 75 DEG C (° C.) to 85 DEG C (° C.); performing reflux and extraction on calculus bovis artifactus with 70% alcohol twice, filtering, combining filtrate obtained twice, recycling alcohol under reduced pressure, and concentrating the filtrate to have a relative density of 1.02 to 1.05 at 75 DEG C (° C.) to 85 DEG C (° C.); decocting the rest of four medicines such as Bulbus Fritillariae Ussuriensis in water, heating and decocting twice, filtering, combining filtrate obtained twice, concentrating the filtrate to have a relative density of 1.10 at 85 DEG C (° C.), centrifuging the filtrate, precipitating liquid supernatant by adding alcohol, standing, taking the liquid supernatant, filtering, recycling alcohol under reduced pressure, and concentrating the filtrate to have a relative density of 1.08 at 85 DEG C); and mixing the concentrated filtrate to obtain the traditional Chinese medicine composition.

The application further discloses a preparation method for the oral liquid, including: uniformly mixing the mixed concentrated filtrate prepared through any preparation method with 0.2% steviosin, adding water to 1000 ml, uniformly mixing, boiling for 40 minutes, refrigerating for not less than 48 h, filtering, adding water into the filtrate until 1000 ml, adjusting a pH value of the filtrate to be 8.0-9.0, filling, and sterilizing to obtain the traditional Chinese medicine composition.

A liquid preparation can be obtained through the preparation method. If a solid preparation needs to be prepared, a pharmaceutical excipients can be added into the liquid medicine and then is prepared into the medicine which is selected from a decoction, a tablet, a capsule, a granule, a pill, an injection, a decocted extract, a suspending agent, a dispersing agent, syrup, a suppository, a gel, aerosol, a patch or an oral liquid.

The oral liquid of the traditional Chinese medicine composition of the present application can have a good protection effect on a VeroE6 cell infected by 2019-nCoV virus under a dilution concentration of 1:10000, 1:20000 or 1:50000, and can inhibit 2019-nCoV replication in cells, so that infected cells have no pathological changes. The traditional Chinese medicine composition of the present application has a good effect in treating or preventing coronavirus related diseases.

### DETAILED DESCRIPTION OF EMBODIMENTS

The present application discloses a use of a traditional Chinese medicine composition, a person skilled in the art appropriately improve the process parameters with reference to the content of this application. It should be noted that all such alternations and modifications are obvious to a person skilled in the art and are considered to be included in the present application. The method of the present application has been described by way of a preferred embodiment, and it is obvious that the method described herein can be modified or appropriately changed and combined to realize and apply the technology of the present application without departing from the scope, spirit and scope of the present application.

Unless otherwise specified, drugs, biomaterials or apparatuses adopted in the present application are common market products, and can be purchased on the market.

The present application is further illustrated in detail in combination with embodiments hereinafter.

### Embodiment 1 Preparation of Traditional Chinese Medicine Oral Liquid

94.5 g of goral horns was cut into threads, an alkaline-containing aqueous solution was used to perform hydrolysis reflux twice, filtrate obtained twice filtrate was combined and concentrated; 15.75 g of Lapis Chloriti and 23.62 g of gypsum were smashed into crude powder, the crude powder was heated and decocted twice in water, filtering was performed, filtrate obtained twice was obtained, and the combined filtrate was concentrated to have a relative density of 1.01 to 1.04 at 75 DEG C (° C.) to 85 DEG C (° C.); reflux and extraction were performed on 9.45 g of calculus bovis artifactus with 70% alcohol twice, filtering was performed, filtrate obtained twice was combined, alcohol was recycled under reduced pressure, and the filtrate was concentrated to have a relative density of 1.02 to 1.05 at 75 DEG C (° C.) to 85 DEG C (° C.); the rest of four medicines: 47.25 g of Bulbus Fritillariae Ussuriensis, 31.5 g of Radix et Rhizoma Rhei, 15.75 parts of radix scutellariae and 31.5 g of radix glycyrrhizae were decocted in water twice, filtering was performed, filtrate obtained twice was combined, the filtrate was concentrated to have a relative density of 1.10 at 85 DEG C (° C.), and was centrifuged, liquid supernatant was precipitated by adding alcohol, standing was performed, the liquid supernatant was taken and filtered, alcohol was recycled under reduced pressure, and the filtrate was concentrated to have a relative density of 1.08 at 85 DEG C (° C.); and the concentrated filtrate was mixed to obtain the traditional Chinese medicine composition.

The concentrated liquor was uniformly mixed with 0.2% steviosin, water was added to 1000 ml, uniformly mixing was performed, boiling was performed for 40 minutes, refrigerating was performed for not less than 48 h, filtering was performed, water was added into the filtrate until 1000 ml, a pH value of the filtrate was adjusted to be 8.0-9.0, filling was performed, and sterilizing was performed to obtain the traditional Chinese medicine composition.

### Efficacy Test Example

1. Experimental Materials

Tested medicines: medicines prepared in Embodiment 1 of the present application.

VeroE6 cell: Stored in Pathogen Center, Institute of Medical Laboratory Animal, Chinese Academy of Medical Sciences.

2019-nCoV virus: with titer of 10⁵TCID₅₀/ml, stored at −80 DEG C (° C.) in Pathogen Center, Institute of Medical Laboratory Animal, Chinese Academy of Medical Sciences. Viral titer used is 100TCID₅₀.

2. Experimental Method

In a sterile 96-well culture plate, 200 μl of a Vero E6 cell with a concentration of 5×10⁴ cell/ml was added into each well, and was cultured with 5% CO₂ for 24 hours at 37 DEG C (° C.); a tested medicine was diluted to have three concentrations: (1:10000, 1:20000 and 1:50000), five wells for each concentration, 100 μl in each well; an equal volume of 100 TCID50 virus was added into each well, and was incubated for 1 h; 1 h later, cell culture fluid in the 96-well culture plate was discarded, and the medicine mixed liquor was added; cell control, blank control (solvent control) and virus control (negative control) were set at the same time; the cell was incubated in an incubator with 5% CO₂ at 37 DEG C (° C.) for 4-5 days. Cytopathic effect (CPE) was observed under an optical microscope, complete cell cytopathy was recorded as “++++”, 75% cell cytopathy was recorded as “+++”, 50% cell cytopathy was recorded as “++”, 25% cell cytopathy was recorded as “+”, and no cell cytopathy was recorded as “−”.

3. Experimental Conditions: The Experimental Operations were all Completed in a BSL-3 Laboratory.

4. Result judgment: when the cell had no CPE, the concentration could effectively inhibit the virus; and when the cell had CPE, the concentration was ineffective.

5. Experimental Results:

The medicine group was set with three concentrations at which 2019-nCoV replication in the cell could be effectively inhibited. The results were shown in table 1.

TABLE 1
Anti-2019-nCoV effect of medicines

| Names of tested | | |
|---|---|---|
| medicines | Concentration | Results |
| Medicines of | 1:10000 | − |
| the present | 1:20000 | − |
| application | 1:50000 | − |
| Negative control/ | | ++++ |
| cytotoxicity | | |

Notes:
“−”indicated no cytopathy, and “++++” indicated complete cell cytopathy.

6. Conclusion:

Based on a cell-level screening result, a sample prepared in Embodiment 1 of the present application could inhibit the 2019-nCoV replication in the cell at 1:10000, 1:20000 and 1:50000 dilution concentrations. This indicated that the medicine in the present application had good in-vitro anti-2019-nCov activity.

It should be noted that a person skilled in the art can properly improve process parameters with reference to the content of this application. All such alternations and modifications are obvious to the person skilled in the art and are considered to be included in the present application.

1-9. (canceled)

10. An application of a traditional Chinese medicine composition in preparing a medicine for treating or preventing coronavirus infection, wherein the traditional Chinese medicine composition is prepared from the following raw materials in parts by weight: 5-15 parts of cornu saigae tataricae or 50-150 parts of goral horns, 10-60 parts of Bulbus Fritillariae Ussuriensis, 15-60 parts of Radix et Rhizoma Rhei, 7-30 parts of radix scutellariae, 7-30 parts of Lapis Chloriti, 10-50 parts of gypsum fibrosum, 5-20 parts of calculus bovis artifactus, and 15-60 parts of radix glycyrrhizae.

11. The application according to claim 10, wherein the coronavirus infection comprises infection of COVID-19.

12. The application according to claim 10, wherein the traditional Chinese medicine composition comprises: 8-12 parts of cornu saigae tataricae or 80-120 parts of goral horns, 15-55 parts of Bulbus Fritillariae Ussuriensis, 20-35 parts of Radix et Rhizoma Rhei, 10-18 parts of radix scutellariae, 10-18 parts of Lapis Chloriti, 15-25 parts of gypsum fibrosum, 8-12 parts of calculus bovis artifactus, and 20-35 parts of radix glycyrrhizae.

13. The application according to claim 10, wherein the traditional Chinese medicine composition comprises: 9.45 parts of cornu saigae tataricae, 17.25 parts of Bulbus Fritillariae Ussuriensis, 31.5 parts of Radix et Rhizoma Rhei, 15.75 parts of radix scutellariae, 15.75 parts of Lapis Chloriti, 23.62 parts of gypsum fibrosum, 9.45 parts of calculus bovis artifactus, and 31.5 parts of radix glycyrrhizae.

14. The application according to claim 10, wherein the traditional Chinese medicine composition comprises: 94.5 parts of goral horns, 47.25 parts of Bulbus Fritillariae Ussuriensis, 31.5 parts of Radix et Rhizoma Rhei, 15.75 parts of radix scutellariae, 15.75 parts of Lapis Chloriti, 23.62 parts of gypsum fibrosum, 9.45 parts of calculus bovis artifactus, and 31.5 parts of radix glycyrrhizae.

15. The application according to claim 10, wherein the medicine for treating or preventing coronavirus infection comprises an oral dosage form, an injection administration form, or an external administration preparation.

16. The application according to claim 15, wherein the medicine for treating or preventing coronavirus infection comprises a decoction, a tablet, a capsule, a granule, a pill, an injection, a decocted extract, a suspending agent, a dispersing agent, a syrup, a suppository, a gel, an aerosol, a patch, or an oral liquid.

17. The application according to claim 10, wherein a method for preparing the traditional Chinese medicine composition comprises the following steps:
taking and cutting the cornu saigae tataricae or goral horns into threads;
hydrolyzing the threads with an alkaline-containing aqueous solution;
filtering, and taking and concentrating filtrate;
taking and smashing the Lapis Chloriti and gypsum fibrosum;
heating and decocting the smashed Lapis Chloriti and gypsum fibrosum in water;
filtering, and taking and concentrating filtrate;
taking and extracting the calculus bovis artifactus with 70% alcohol;
filtering, and concentrating filtrate;
decocting a rest of medicines in water;
concentrating and centrifuging filtrate;
precipitating liquid supernatant with alcohol;
standing, taking the liquid supernatant, filtering, and concentrating filtrate; and
mixing the concentrated filtrate to obtain the traditional Chinese medicine composition.

18. The application according to claim 17, wherein the cornu saigae tataricae or goral horns are cut into threads, the alkaline-containing aqueous solution is used to perform hydrolysis reflux twice, and filtrate obtained twice is concentrated; the Lapis Chloriti and gypsum fibrosum are smashed into crude powder, the crude powder is heated and decocted twice in water, filtering is performed, filtrate obtained twice is combined, and the combined filtrate is concentrated to have a relative density of 1.01 to 1.04 at 75° C. to 85° C.; reflux and extraction is performed on calculus bovis artifactus with 70% alcohol twice, filtering is performed, filtrate obtained twice is combined, alcohol is recycled under reduced pressure, and the combined filtrate is concentrated to have a relative density of 1.02 to 1.05 at 75° C. to 85° C.; the rest of medicines are decocted in water twice, and filtered, and filtrate obtained twice is combined, the combined filtrate is concentrated to have a relative density of 1.10 at 85° C., and is centrifuged, liquid supernatant is precipitated by adding alcohol, and is taken and filtrated after standing is performed, alcohol is recycled under reduced pressure, and then filtrate is concentrated to have a relative density of 1.08 at 85° C.; and the concentrated filtrate is mixed to obtain the traditional Chinese medicine composition.
